(19) United States
(12) Patent Application Publication
(10) Pub. No.: US 2026/0085043 A1
(43) Pub. Date: Mar. 26, 2026

(54) INITIATOR MIXTURE, COMPOSITION INCLUDING THE SAME, AND POLYMER FORMED FROM THE COMPOSITION

(71) Applicants: Samsung Display Co., Ltd., Yongin-si (KR); Seoul National University R&DB Foundation, Seoul (KR)

(72) Inventors: Shinya Onoue, Yongin-si (KR); Minsang Kwon, Seoul (KR); Daehyun Hwang, Yongin-si (KR); Yonghwan Kwon, Seoul (KR); Seokju Lee, Seoul (KR); Hyeondeuk Hwang, Yongin-si (KR)

(21) Appl. No.: 19/333,173

(22) Filed: Sep. 18, 2025

(30) Foreign Application Priority Data
Sep. 24, 2024 (KR) 10-2024-0129428

(51) Int. Cl. C07D 209/86; C07C 49/303; C07C 211/54; C07C 233/03; C07F 5/02

(52) U.S. Cl. CPC C07D 209/86; C07C 49/303; C07C 211/54; C07C 233/03; C07F 5/027

(57) ABSTRACT

Provided is an initiator mixture including a multi-aromatic ring-containing compound, an ionic compound including iodine, an ionic compound including boron, a compound including a norbornene moiety which includes an oxo group (O═), and a tertiary amine compound including an aromatic moiety, an ester group, and an amine group.

### CROSS-REFERENCE TO RELATED APPLICATION

The present application claims priority to and the benefit of Korean Patent Application No. 10-2024-0129428, filed on Sep. 24, 2024, in the Korean Intellectual Property Office, the entire content of which is hereby incorporated by reference.

### BACKGROUND

### 1. Field

One or more embodiments of the present disclosure relate to an initiator mixture, a composition including the same, and a polymer formed from the composition.

### 2. Description of the Related Art

Polymers prepared through thermosetting have problems in that the manufacturing time is relatively long, processes including molding are complex, and investment costs are increased due to the complex processes.

Although polymers prepared through photocuring have an advantage in terms of taking a relatively short time, there is a limitation in a wavelength range and/or the like of light used for photocuring.

### SUMMARY

One or more embodiments of the present disclosure include an initiator mixture and/or the like.

Additional aspects of embodiments will be set forth in part in the description which follows and, in part, will be apparent from the description, or may be learned by practice of the presented embodiments of the disclosure.

According to one or more embodiments, an initiator mixture includes a multi-aromatic ring-containing compound, an ionic compound including iodine, an ionic compound including boron, a compound including a norbornene moiety which includes an oxo group (O═), and a tertiary amine compound including an aromatic moiety, an ester group, and an amine group.

According to one or more embodiments, a composition incudes the initiator mixture and a monomer.

According to one or more embodiments, a polymer is formed by irradiating visible light onto the composition.

### BRIEF DESCRIPTION OF THE DRAWINGS

The above and other aspects and features of certain embodiments will be more apparent from the following description taken in conjunction with the accompanying drawings, in which:

FIG. 1 is a graph showing conversion rates over time of compositions of comparative examples and an example;

FIG. 2 shows images showing results of evaluating deep curing over time of the compositions of comparative examples and an example; and

FIG. 3 shows graphs showing results of evaluating deep curing over time of compositions of comparative examples and an example.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the FIGS., to explain aspects of embodiments of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

Although polymers prepared through photocuring have an advantage or feature in terms of taking a relatively short time (e.g., in terms of curing in a relatively short period of time), there is a limitation in wavelength range and/or the like of light used for photocuring.

For example, when a composition is cured by ultraviolet (UV) rays, photocuring is difficult when a layer (for example, a polyimide film) that blocks (or reduces transmission of) UV rays is present on the composition.

In order to further improve the physical properties of polymers using a curing system according to a related art, it is necessary to increase a conversion rate.

After a long study, the inventors have developed a new initiator mixture that can be used as a new curing system.

An initiator mixture according to an aspect of an embodiment may include a multi-aromatic ring-containing compound, an ionic compound including iodine, an ionic compound including boron, a compound including a norbornene moiety which includes an oxo group (O═), and a tertiary amine compound including an aromatic moiety, an ester group, and an amine group.

According to an embodiment, the initiator mixture may generate radicals in response to visible light. For example, a wavelength of the visible light may be a wavelength of blue light and/or a wavelength of green light. For example, the initiator mixture may generate radicals upon exposure to light having a wavelength of about 400 nm to about 500 nm.

The multi-aromatic ring-containing compound in the initiator mixture refers to a compound including a plurality of aromatic rings. For example, the multi-aromatic ring-containing compound may include a compound including nine or more phenyl moieties.

According to an embodiment, the multi-aromatic ring-containing compound may include a first compound represented by Formula 1 below:

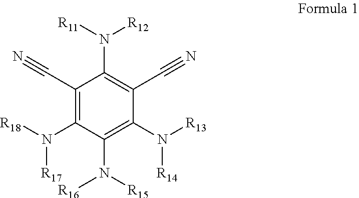

R₁₁ to R₁₈ may each independently be hydrogen, deuterium, —F, —Cl, —Br, —I, a hydroxyl group, a cyano group, a nitro group, a C₁-C₆₀ alkyl group unsubstituted or substituted with at least one R^{10a}, a C₂-C₆₀ alkenyl group unsubstituted or substituted with at least one R^{10a}, a C₂-C₆₀ alkynyl group unsubstituted or substituted with at least one R^{10a}, a C₁-C₆₀ alkoxy group unsubstituted or substituted with at least one R^{10a}, a C₃-C₆₀ carbocyclic group unsubstituted or substituted with at least one R^{10a}, a C₁-C₆₀ heterocyclic group unsubstituted or substituted with at least one R^{10a}, a C₁-C₆₀ aryloxy group unsubstituted or substituted with at least one R^{10a}, a C₆-C₆₀ arylthio group unsubstituted or substituted with at least one R^{10a}, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more adjacent ones of R₁₁ to R₁₈ may be optionally linked to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
R₁₀ₐ may be: deuterium, —F, —Cl, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof; a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently: be hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to an embodiment, the first compound may be represented by Formula 1-1 or Formula 1-2 below:

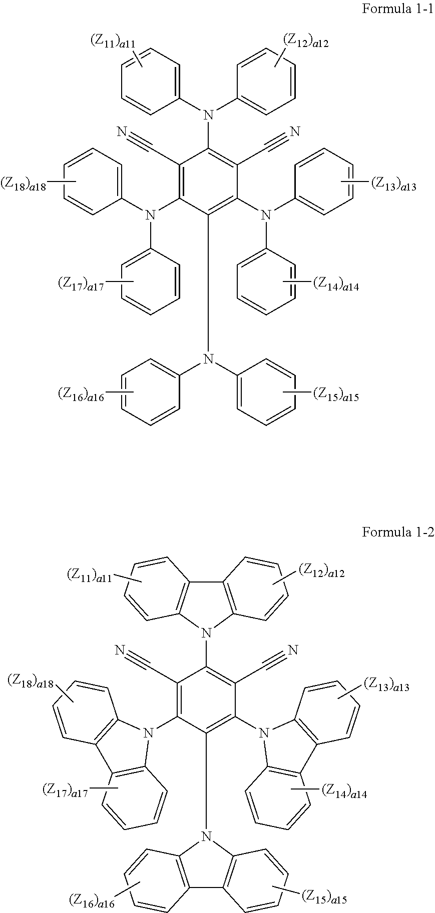

In Formula 1-1 and Formula 1-2,
Z₁₁ to Z₁₈ may each independently be hydrogen or defined as for R₁₀ₐ, and
a11 to a18 may each independently be an integer from 1 to 4.

According to an embodiment, the first compound may include at least one selected from compounds below:

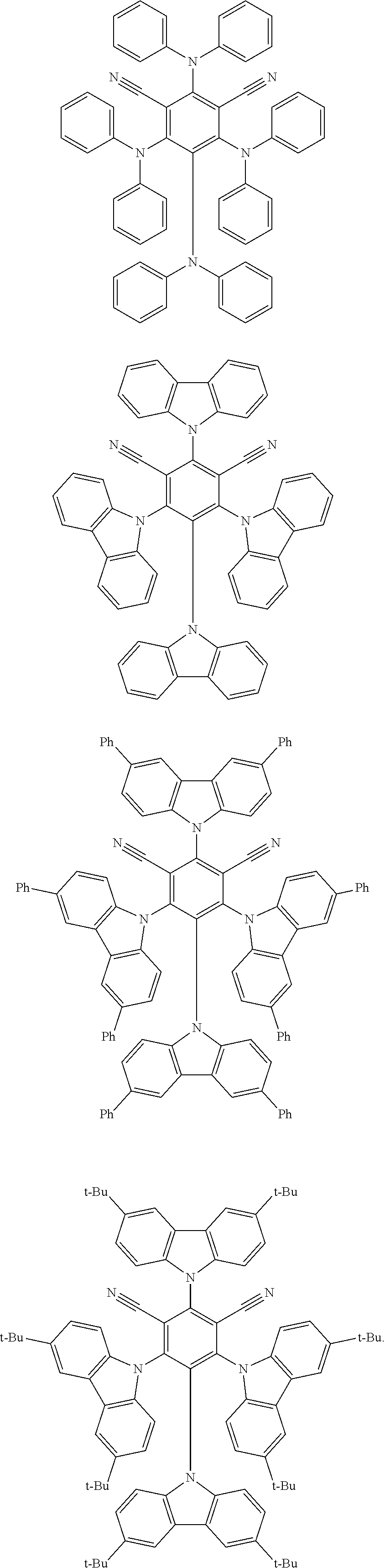

According to an embodiment, the ionic compound including iodine may include a second compound represented by Formula 2 below:

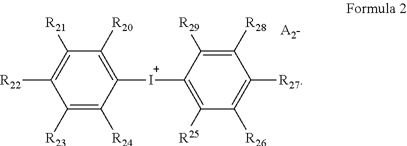

In Formula 2,
A₂_{^{−}} may be a counter anion,
R₂₀ to R₂₉ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more adjacent ones of R₂₀ to R₂₉ may be optionally linked to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
R₁₀ₐ may be: deuterium, —F, —Cl, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof; a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to an embodiment, A₂_{^{−}} may include a fluorine atom.

According to an embodiment, the ionic compound including iodine may include at least one selected from compounds below:

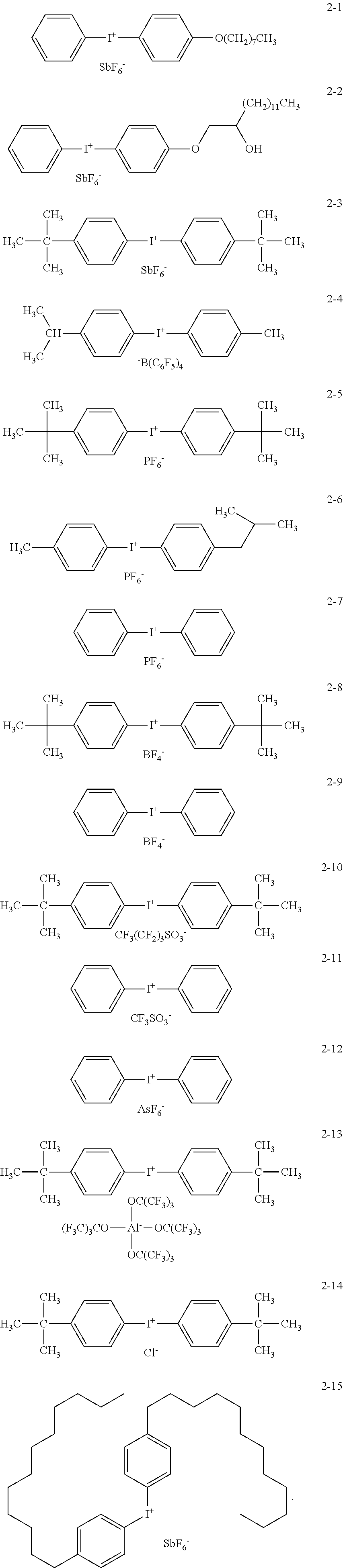

According to an embodiment, the ionic compound including boron may include a third compound represented by Formula 3 below:

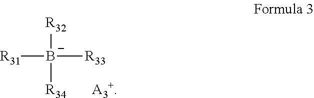

In Formula 3,
A₃_{⁺} may be a counter cation,
R₃₁ to R₃₄ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more adjacent ones of R₃₁ to R₃₄ may be optionally linked to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
R₁₀ₐ may be: deuterium, —F, —Cl, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof; a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to an embodiment, A₃_{⁺} may be [P(R₃₁ₐ)(R_{31b})(R_{31c})(R_{31d})]⁺ or [N(R₃₂ₐ)(R_{32b})(R_{32c})(R_{32d})]⁺.

R₃₁ₐ, R_{31b}, R_{31c}, R_{31d}, R₃₂ₐ, R_{32b}, R_{32c}, and R_{32d} may each independently be a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ.

R₁₀ₐ may be as defined above.

According to an embodiment, the ionic compound including boron may include at least one selected from compounds below:

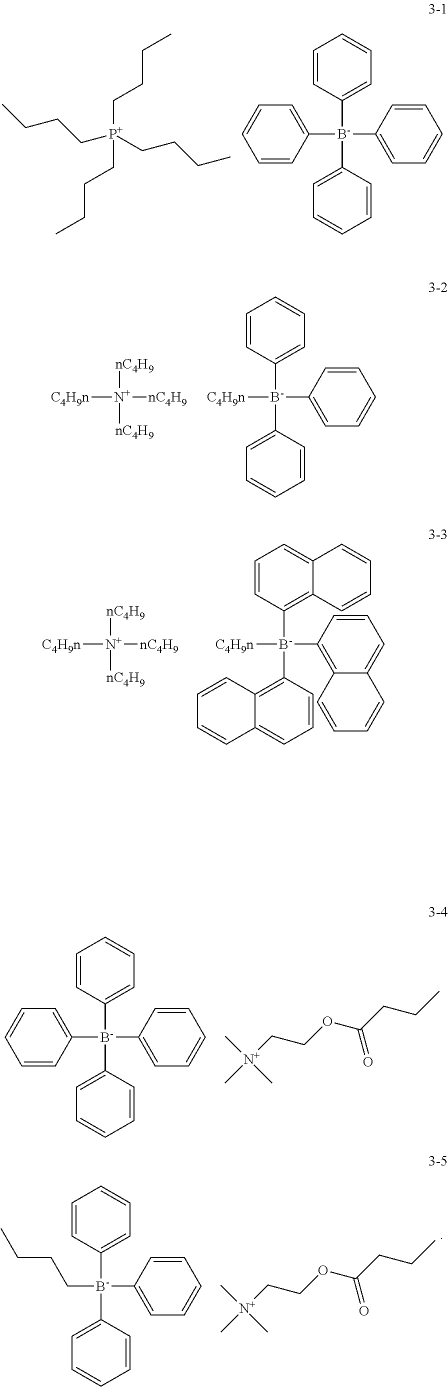

According to an embodiment, the norbornene moiety including the oxo group (O═) may include 2-oxobicyclo[2.2.1]heptane.

For example, the compound including the norbornene moiety which includes the oxo group (O═) may include a fourth compound represented by Formula 4 below:

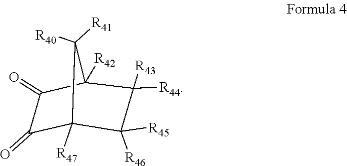

In Formula 4,
R₄₀ to R₄₇ may each independently be hydrogen, deuterium, —F, —Cl, —Br, —I, a hydroxyl group, a cyano group, a nitro group, a C₁-C₆₀ alkyl group unsubstituted or substituted with at least one R^{10a}, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
R₁₀ₐ and Q₁ to Q₃ may be as defined above.

According to an embodiment, the compound including the norbornene moiety which includes the oxo group (O═) may include camphorquinone (CQ), 7,7-dimethyl-2,3-dioxobicyclo[2.2.1]heptane-1-carboxylic acid, 7,7-dimethyl-2,3-dioxobicyclo[2.2.1]heptane-1-carboxy-2-bromoethyl ester, 7,7-dimethyl-2,3-dioxobicyclo[2.2.1]heptane-1-carboxy-2-methyl ester, 7,7-dimethyl-2,3-dioxobicyclo[2.2.1]heptane-1-carboxylic acid chloride, or any combination thereof.

According to an embodiment, the aromatic moiety of the tertiary amine compound may include a phenyl group.

According to an embodiment, the aromatic moiety of the tertiary amine compound may be a phenyl group, and the ester group and the amine group may be linked to the phenyl group and may be at para positions of the phenyl group.

According to an embodiment, the tertiary amine compound including the aromatic moiety, the ester group, and the amine group may include a fifth compound represented by Formula 5 below:

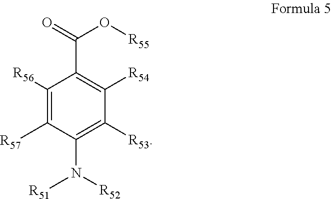

In Formula 5,
R₅₁ to R₅₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), and
R₁₀ₐ and Q₁ to Q₃ may be as defined above.

According to an embodiment, the tertiary amine compound including the aromatic moiety, the ester group, and the amine group may include at least one selected from compounds below:

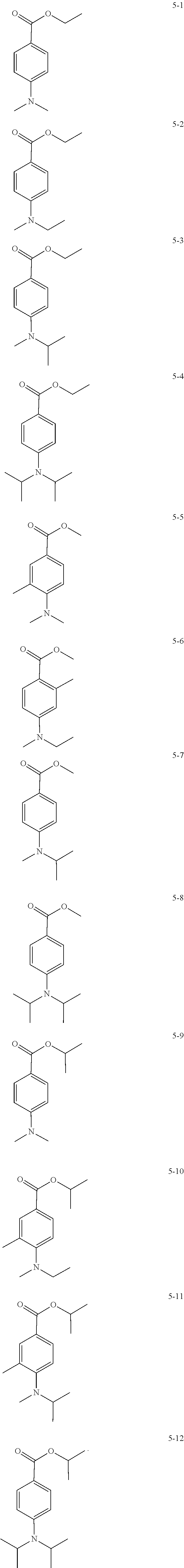

A composition according to another aspect of embodiments may include the initiator mixture and a monomer.

According to an embodiment, a content (e.g., amount) of the multi-aromatic ring-containing compound may be greater than 0 ppm and less than or equal to 100 ppm (with respect to the total amount of the composition). For example, the content (e.g., amount) of the multi-aromatic ring-containing compound may be in a range of about 9 parts per million (ppm) to about 15 ppm. The range of the content (e.g., amount) of the multi-aromatic ring-containing compound may be consistent with a content (e.g., amount) of the fourth compound to be further described herein below and thus may be suitable, and the physical properties of the polymer formed using the initiator mixture may be excellent.

According to an embodiment, a content (e.g., amount) of the ionic compound including iodine may be in a range of about 500 ppm to about 6,000 ppm (with respect to the total amount of the composition).

According to an embodiment, a content (e.g., amount) of the ionic compound including boron may be in a range of about 600 ppm to about 4,000 ppm (with respect to the total amount of the composition).

For example, the content (e.g., amount) of the ionic compound including iodine may be in a range of about 2,000 ppm to about 5,000 ppm, and the content (e.g., amount) of the ionic compound including boron may be in a range of about 1,000 ppm to about 3,000 ppm.

When the content (e.g., amount) of the ionic compound including iodine and the content (e.g., amount) of the ionic compound including boron are within the above ranges, radicals utilized or required for curing the composition according to an embodiment may be continuously (e.g., substantially continuously) generated in accordance with the content (e.g., amount) of the multi-aromatic ring-containing compound.

According to an embodiment, a content (e.g., amount) of the compound including the norbornene moiety which includes the oxo group (O═) may be in a range of about 1,000 ppm to about 4,000 ppm (with respect to the total amount of the composition).

According to an embodiment, a content (e.g., amount) of the tertiary amine compound including the aromatic moiety, the ester group, and the amine group may be 5 to 7 times the content (e.g., amount) of the compound including the norbornene moiety which includes the oxo group (O═).

For example, the content (e.g., amount) of the fourth compound, which is the compound including the norbornene moiety which includes the oxo group (O═), may be in a range of about 1,500 ppm to about 4,000 ppm, and a content (e.g., amount) of the fifth compound, which is the tertiary amine compound including the aromatic moiety, the ester group, and the amine group, may be about 6 times the content (e.g., amount) of the compound including the norbornene moiety which includes the oxo group (O═).

When the contents (e.g., amounts) of the first to fifth compounds in the initiator mixture are each within the above range, the physical properties of the polymer formed using the same may be excellent.

In embodiments, the total content (e.g., amount) of respective compounds in the initiator mixture may be 20,000 ppm or less. When the total content (e.g., amount) of the initiator mixture is up to 20,000 ppm, the total content (e.g., amount) may correspond to 2 wt % with respect to the composition and may be less than an initiator content (e.g., amount) of about 3 wt % to about 10 wt % used in a related art (for example, an acrylic resin composition).

Because each of the compounds in the initiator mixture is decomposed during a reaction process, there is an advantage or feature in that an amount of outgas is relatively small.

According to an embodiment, the monomer may include an acrylic monomer.

According to an embodiment, a content (e.g., amount) of the acrylic monomer may be in a range of about 1 wt % to about 50 wt %. For example, the content (e.g., amount) of the acrylic monomer may be in a range of about 5 wt % to about 30 wt %. When the content (e.g., amount) of the acrylic monomer deviates from the above range, the physical properties of a formed polymer may not be excellent or suitable.

According to an embodiment, the acrylic monomer may include a C₁-C₂₀ alkyl group, a C₃-C₁₀ cycloalkyl group, —NCO, —OH, or any combination thereof.

For example, the acrylic monomer may include a monofunctional (meth)acrylate, a polyfunctional (meth)acrylate, or a combination thereof. The monofunctional (meth)acrylate may include methyl (meth)acrylate, ethyl (meth)acrylate, propyl (meth)acrylate, butyl (meth)acrylate, 2-ethylhexyl (meth)acrylate, isooctyl (meth)acrylate, isodecyl (meth)acrylate, lauryl (meth)acrylate, stearyl (meth)acrylate, phenyl (meth)acrylate, cyclohexyl (meth)acrylate, dicyclopentanyl (meth)acrylate, dicyclopentenyloxyethyl (meth)acrylate, isobornyl (meth)acrylate, methoxylated cyclodecatriene (meth)acrylate, 2-hydroxyethyl (meth)acrylate, 2-hydroxypropyl (meth)acrylate, 3-hydroxypropyl (meth)acrylate, 4-hydroxybutyl (meth)acrylate, tetrahydrofurfuryl (meth)acrylate, 2-hydroxy-3-phenoxypropyl (meth)acrylate, glycidyl (meth)acrylate, caprolactone-modified tetrahydrofurfuryl (meth)acrylate, 3-chloro-2-hydroxypropyl (meth)acrylate, N,N-dimethylaminoethyl (meth)acrylate, N,N-siethylaminoethyl (meth)acrylate, t-butylaminoethyl (meth)acrylate, ethoxycarbonylmethyl (meth)acrylate, phenol ethylene oxide-modified acrylate, phenol (2-mol ethylene oxide-modified) acrylate, phenol (4-mol ethylene oxide-modified) acrylate, paracumylphenol ethylene oxide-modified acrylate, nonylphenol ethylene oxide-modified acrylate, nonylphenol (4-mol ethylene oxide-modified) acrylate, nonylphenol (8-mol ethylene oxide-modified) acrylate, nonylphenol (2.5-mol propylene oxide-modified) acrylate, 2-ethylhexyl carbitol acrylate, ethylene oxide-modified phthalic acid (meth)acrylate, ethylene oxide-modified succinic acid (meth)acrylate, trifluoroethyl (meth)acrylate, an acrylic acid, a methacrylic acid, a maleic acid, a fumaric acid, ω-carboxy-polycaprolactone mono(meth)acrylate, phthalic acid monohydroxyethyl (meth)acrylate, a (meth)acrylic acid dimer, β-(meth)acroyloxyethyl hydrogen succinate, n-(meth)acryloyloxyalkyl hexahydrophthalimid, a hydrophilic group substituent thereof, or a combination thereof.

The polyfunctional (meth)acrylate may include a difunctional (meth)acrylate, a trifunctional (meth)acrylate, a tetrafunctional or higher (meth)acrylate, or a combination thereof.

The difunctional (meth)acrylate may 1,3-butylene glycol di(meth)acrylate, 1,4-butanediol di(meth)acrylate, 1,6-hexadiol di(meth)acrylate, 1,9-nonanediol di(meth)acrylate, neopentyl glycol di(meth)acrylate, dicyclopentanyl di(meth)acrylate, 2-ethyl-2-butyl-propanediol (meth)acrylate, neopentyl glycol-modified trimethylolpropane di(meth)acrylate, stearic acid-modified pentaerythritol diacrylate, polypropylene glycol di(meth)acrylate, 2,2-bis(4-(meth)acryloxy diethoxyphenyl)propane, 2,2-bis(4-(meth)acryloxy propoxyphenyl)propane or 2,2-bis(4-(meth)acryloxy tetraethoxyphenyl)propane, a hydrophilic group substituent thereof, or a combination thereof.

The trifunctional (meth)acrylate may include trimethylolpropane tri(meth)acrylate, tris[(meth)acryloxyethyl]isocyanurate, a hydrophilic group substituent thereof, or a combination thereof.

The tetrafunctional or higher (meth)acrylate may include dimethylolpropane tetra(meth)acrylate, pentaerythritol tetra(meth)acrylate, pentaerythritolethoxy tetra(meth)acrylate, dipentaerythritol penta(meth)acrylate. dipentaerythritol hexa(meth)acrylate, a hydrophilic group substituent thereof, or a combination thereof.

In the composition according to an embodiment, excluding the content (e.g., amount) of the acrylic monomer, the content (e.g., amount) of the multi-aromatic ring-containing compound, and the contents (e.g., amounts) of the second to fifth compounds, the remainder may include other monomers (with respect to 100 wt % of the total amount of the composition).

According to an embodiment, the other monomers may include a diisocyanate-based monomer.

According to an embodiment, the other monomers may include 2,4-toluene diisocyanate, isophorone diisocyanate, hexamethylene diisocyanate, methylene diphenyl diisocyanate, or any combination thereof.

In the composition according to an embodiment, excluding the content (e.g., amount) of the monomer, the content (e.g., amount) of the multi-aromatic ring-containing compound, and the contents (e.g., amounts) of the second to fifth compounds, the remainder may include other monomers and a solvent (with respect to 100 wt % of the total amount of the composition). The solvent may include, for example, n-hexane, toluene, xylene, acetone, ethanol, isopropyl alcohol, or any combination thereof.

According to another aspect of embodiments, there may be provided a polymer formed by irradiating visible light onto the composition.

For example, the polymer according to an embodiment may be formed by curing the composition for about 0.1 seconds to about 20 minutes with visible light. For example, the polymer according to an embodiment may be formed by curing the composition for about 10 seconds to about 10 minutes with visible light.

According to an embodiment, a peak top of a wavelength of the irradiated visible light may be in a range of about 400 nm to about 560 nm. For example, the peak top of the wavelength of the visible light may be in a range of about 430 nm to about 490 nm. For example, the peak top of the wavelength of the visible light may be in a range of about 450 nm to about 480 nm.

### DEFINITION OF TERMS

As used herein, the term “C₃-C₆ carbocyclic group” refers to a cyclic group having 3 to 60 carbon atoms and consisting only of carbon as a ring-forming atom, and the term “C₁-C₆₀ heterocyclic group” refers to a cyclic group having 1 to 60 carbon atoms and further including a heteroatom as a ring-forming atom in addition to carbon. Each of the C₃-C₆₀ carbocyclic group and the C₁-C₆₀ heterocyclic group may be a monocyclic group having one ring or a polycyclic group in which two or more rings are condensed with each other. For example, the number of ring-forming atoms of the C₁-C₆₀ heterocyclic group may be in a range of 3 to 61.

As used herein, the term “cyclic group” includes both the C₃-C₆₀ carbocyclic group and the C₁-C₆₀ heterocyclic group.

As used herein, the term “π electron-rich C₃-C₆₀ cyclic group” refers to a cyclic group that has 3 to 60 carbon atoms and does not include *—N═*′ as a ring-forming moiety, and the term” π electron-deficient nitrogen-containing C₁-C₆₀ cyclic group” refers to a heterocyclic group that has 1 to 60 carbon atoms and includes *—N═*′ as a ring-forming moiety.

For example, the C₃-C₆₀ carbocyclic group may be i) a group T1 or ii) a condensed ring group in which two or more groups T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group).

The C₁-C₆₀ heterocyclic group may be i) a group T2, ii) a fused ring group in which two or more groups T2 are fused with each other, or iii) a fused ring group in which one or more groups T2 and one or more groups T1 are fused with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group).

The π electron-rich C₃-C₆₀ cyclic group may be i) a group T1, ii) a fused ring group in which two or more groups T1 are fused with each other, iii) a group T3, iv) a fused ring group in which two or more groups T3 are fused with each other, or v) a fused ring group in which one or more groups T3 and one or more groups T1 are fused with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, or a benzothienodibenzothiophene group).

The π electron-deficient nitrogen-containing C₁-C₆₀ cyclic group may be i) a group T4, ii) a fused ring group in which two or more group T4 are fused with each other, iii) a fused ring group in which one or more groups T4 and one or more groups T1 are fused with each other, iv) a fused ring group in which one or more groups T4 and one or more groups T3 are fused with each other, or v) a fused ring group in which one or more groups T4G, one or more groups T1, and one or more groups T3 are fused with each other (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group).

The group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group.

The group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

The group T3 may be a furan group, a thiophene group, a 1H-pyrrole group, a silole group, or a borole group.

The group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

As used herein, the term “cyclic group,” “C₃-C₆₀ carbocyclic group,” “C₁-C₆₀ heterocyclic group,” “π electron-rich C₃-C₆₀ cyclic group,” or “π electron-deficient nitrogen-containing C₁-C₆₀ cyclic group” may be a group condensed to an arbitrary cyclic group, a monovalent group, or a multivalent group (for example, a divalent group, a trivalent group, or a tetravalent group) according to a structure of a formula in which the term is used. For example, a “benzene group” may be a benzo group, a phenyl group, a phenylene group, or the like, which may be understood by those skilled in the art according to a structure of a formula including the “benzene group.”

For example, examples of a monovalent C₃-C₆₀ carbocyclic group and a monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed hetero-polycyclic group, and examples of a divalent C₃-C₆₀ carbocyclic group and a divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed hetero-polycyclic group.

As used herein, the term “C₁-C₆₀ alkyl group” refers to a linear or branched aliphatic hydrocarbon monovalent group having 1 to 60 carbon atoms, and specific examples thereof include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and the like. As used herein, the term “C₁-C₆₀ alkylene group” refers to a divalent group having substantially the same structure as the C₁-C₆₀ alkyl group.

As used herein, the term “C₂-C₆₀ alkenyl group” refers to a monovalent hydrocarbon group including one or more carbon-carbon double bonds at a main chain (e.g., in the middle) or a terminal end (e.g., a terminus) of a C₂-C₆₀ alkyl group, and examples thereof include an ethenyl group, a propenyl group, a butenyl group, and the like. As used herein, the term “C₂-C₆₀ alkenyl group” refers to a divalent group having substantially the same structure as the C₂-C₆₀ alkenyl group.

As used herein, the term “C₂-C₆₀ alkynyl group” refers to a monovalent hydrocarbon group including one or more carbon-carbon triple bonds at a main chain (e.g., in the middle) or a terminal end (e.g., a terminus) of a C₂-C₆₀ alkyl group, and specific examples thereof include an ethynyl group, a propynyl group, and the like. As used herein, the term “C₂-C₆₀ alkynylene group” refers to a divalent group having substantially the same structure as the C₂-C₆₀ alkynyl group.

As used herein, the term “C₁-C₆₀ alkoxy group” refers to a monovalent group having a formula of —OA₁₀₁, wherein A₁₀₁ is the C₁-C₆₀ alkyl group, and examples thereof include a methoxy group, an ethoxy group, an isopropyloxy group, and the like.

As used herein, the term “C₃-C₁₀ cycloalkyl group” refers to a monovalent saturated hydrocarbon cyclic group having 3 to 10 carbon atoms, and specific examples thereof include a cyclopropyl group, a cyclobutyl group, a cyclopentyl group, a cyclohexyl group, a cycloheptyl group, a cyclooctyl group, adamantanyl, a norbornanyl group (or, a bicyclo[2.2.1]heptyl group), a bicyclo[1.1.1]pentyl group, a bicyclo[2.1.1]hexyl group, a bicyclo[2.2.2]octyl group, and the like. As used herein, the term “C₃-C₁₀ cycloalkylene group” refers to a divalent group having substantially the same structure as the C₃-C₁₀ cycloalkyl group.

As used herein, the term “C₁-C₁₀ heterocycloalkyl group” refers to a monovalent cyclic group having 1 to 10 carbon atoms and further including at least one heteroatom as a ring-forming atom in addition to carbon atoms, and examples thereof include a 1,2,3,4-oxatriazolidinyl group, a tetrahydrofuranyl group, a tetrahydrothiophenyl group, and the like. As used herein, the term “C₁-C₁₀ heterocycloalkylene group” refers to a divalent group having substantially the same structure as the C₁-C₁₀ heterocycloalkyl group.

As used herein, the term “C₃-C₁₀ cycloalkenyl group” is a monovalent cyclic group which has 3 to 10 carbon atoms and refers to a group having at least one carbon-carbon double bond in a ring thereof but not having aromaticity (e.g., is not aromatic), and examples thereof include a cyclopentenyl group, a cyclohexenyl group, a cycloheptenyl group, and the like. As used herein, the term “C₃-C₁₀ cycloalkenylene group” refers to a divalent group having substantially the same structure as the C₃-C₁₀ cycloalkenyl group.

As used herein, the term “C₁-Cia heterocycloalkenyl group” is a monovalent cyclic group which has 1 to 10 carbon atoms, further includes at least one heteroatom as a ring-forming atom in addition to carbon atoms, and has at least one double bond in a ring thereof. Examples of the C₁-Cia heterocycloalkenyl group include a 4,5-dihydro-1,2,3,4-oxatriazolyl group, a 2,3-dihydrofuranyl group, a 2,3-dihydrothiophenyl group, and the like. As used herein, the term “C₁-C₁₀ heterocycloalkenylene group” refers to a divalent group having substantially the same structure as the C₁-Cia heterocycloalkenyl group.

As used herein, the term “C₆-C₆₀ aryl group” refers to a monovalent group having a carbocyclic aromatic system which has 6 to 60 carbon atoms, and the term “C₆-C₆₀ arylene group” refers to a divalent group having a carbocyclic aromatic system which has 6 to 60 carbon atoms. Examples of the C₆-C₆₀ aryl group include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and the like. When the C₆-C₆₀ aryl group and the C₆-C₆₀ arylene group include two or more rings, the two or more rings may be condensed with each other.

As used herein, the term “C₁-C₆₀ heteroaryl group” refers to a monovalent group further including at least one heteroatom as a ring-forming atom in addition to carbon atoms and having a heterocyclic aromatic system which has 1 to 60 carbon atoms, and the C₁-C₆₀ heteroarylene group refers to a divalent group further including at least one heteroatom as a ring-forming atom in addition to carbon atoms and having a heterocyclic aromatic system which has 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and the like. When the C₁-C₆₀ heteroaryl group and the C₁-C₆₀ heteroarylene group include two or more rings, the two or more rings may be condensed with each other.

As used herein, the term “monovalent non-aromatic condensed polycyclic group” refers to a monovalent group (for example, having 8 to 60 carbon atoms) in which two or more rings are condensed with each other, which includes only carbon as a ring-forming atom, and of which the entire molecule has non-aromaticity (e.g., is not aromatic when considered as a whole). Examples of the monovalent non-aromatic condensed polycyclic group include an indenyl group, a fluorenyl group, a spiro-bifluorenyl group, a benzofluorenyl group, an indenophenanthrenyl group, an indenoanthracenyl group, and the like. As used herein, the term “divalent non-aromatic condensed polycyclic group” refers to a divalent group having substantially the same structure as the monovalent non-aromatic condensed polycyclic group.

As used herein, the term “monovalent non-aromatic condensed hetero-polycyclic group” refers to a monovalent group (for example, having 1 to 60 carbon atoms) in which two or more rings are condensed with each other, which further includes at least one heteroatom as a ring-forming atom in addition to carbon atoms, and of which the entire molecule has non-aromaticity (e.g., is not aromatic when considered as a whole). Examples of the monovalent non-aromatic condensed hetero-polycyclic group include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and the like. As used herein, the term “divalent non-aromatic condensed hetero-polycyclic group” refers to a divalent group having the same structure as the monovalent non-aromatic condensed hetero-polycyclic group.

As used herein, the term “C₆-C₆₀ aryloxy group” refers to —OA₁₀₂, wherein A₁₀₂ is the C₆-C₆₀ aryl group, and the C₆-C₆₀ arylthio group refers to —SA₁₀₃, wherein A₁₀₃ is the C₆-C₆₀ aryl group.

As used herein, the term “C₇-C₆₀ arylalkyl group” refers to -A₁₀₄A₁₀₅, wherein A₁₀₄ is a C₁-C₅₄ alkylene group and A₁₀₅ is a C₆-C₅₉ aryl group, and as used herein, the term “C₂-C₆₀ heteroarylalkyl group” refers to -A₁₀₆A₁₀₇, wherein A₁₀₆ is a C₁-C₅₉ alkylene group and A₁₀₇ is a C₁-C₅₉ heteroaryl group.

As used herein, “R₁₀ₐ” may be: deuterium, —F, —CI, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —CI, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof; a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —CI, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

As used herein, Q₁ to Q₃, Q₁₁ to Q₁₃, Q₂₁ to Q₂₃, and Q₃₁ to Q₃₃ may each independently be: hydrogen; deuterium; —F; —CI; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

As used herein, the term “heteroatom” refers to any atom other than a carbon atom. Examples of the heteroatom include O, S, N, P, Si, B, Ge, Se, or any combination thereof.

As used herein, the term “third-row transition metal includes hafnium (Hf), tantalum (Ta), tungsten (W), rhenium (Re), osmium (Os), iridium (Ir), platinum (Pt), gold (Au), or the like.

As used herein, the term “Ph” refers to a phenyl group, the term “Me” refers to a methyl group, the term “Et” refers to an ethyl group, the term “tert-Bu” or “Bu^{t}” refers to a tert-butyl group, and the term “OMe” refers to a methoxy group.

As used herein, the term “biphenyl group” refers to a “phenyl group substituted with a phenyl group.” The “biphenyl group” belongs to a “substituted phenyl group” of which a substituent is a “C₆-C₆₀ aryl group.”

As used herein, the term “terphenyl group” refers to a “phenyl group substituted with a biphenyl group.” The “terphenyl group” belongs to a “substituted phenyl group” of which a substituent is a “C₆-C₆₀ aryl group substituted with a C₆-C₆₀ aryl group.”

In the definition of a substituent, the number of carbon atoms merely an example. For example, in a C₁-C₆₀ alkyl group, C₆₀ is merely an example, and the definition of an alkyl group is equally applied to a C₁-C₂₀ alkyl group. The same may be applied to other embodiments and definitions herein.

Any hydrogen in a compound structure of the present specification may be optionally substituted with deuterium.

Spatially relative terms, such as “below,” “beneath,” “lower,” “above,” “upper,” and the like, may be used herein to easily describe a relationship between one device or components and other devices or components as illustrated in the drawings. Spatially relative terms are to be understood to include different orientations of a device when in use or operation in addition to the orientation illustrated in the drawings. For example, when a device shown in the drawings is turned over, devices described as “below” or “beneath” other devices may be “above” the other devices. Thus, the example term “below” may encompass both orientations of above and below. A device may be otherwise oriented, and thus spatially relative terms may be interpreted according to orientation.

Hereinafter, a composition according to an embodiment will be described in more detail with reference to Examples.

### EXAMPLES

### Composition Preparation

### Comparative Example 1

4,000 ppm of CQ and 24,000 ppm of ethyl 4-(dimethylamino)benzoate [EDB] were used as an initiator mixture, isobornyl acrylate (IBOA) was mixed as the remainder (with respect to 100 wt % of the total amount of the composition), thereby preparing a composition.

Contents (e.g., amounts) of the CQ and the EDB are selected as optimal contents (e.g., amounts) from among various suitable contents (e.g., amounts) of the CQ and the EDB.

### Comparative Example 2

25 ppm of 4DP-IPN and 3,000 ppm of compound 2-4, and 1,800 ppm of compound 3-2 were used as an initiator mixture, IBOA was mixed as the remainder (with respect to 100 wt % of the total amount of the composition), thereby preparing a composition.

Contents (e.g., amounts) of the 4DP-IPN, compound 2-4, and compound 3-2 are optimal contents (e.g., amounts) selected from among various suitable contents (e.g., amounts) of the 4DP-IPN, compound 2-4, and compound 3-2.

### Example 1

Predetermined amounts of CQ, EDB, 4DP-IPN, compound 2-4, and compound 3-2 were used as an initiator mixture, and bis-A type epoxy methacrylate (Bis A GMA) and 1,3-dimethylamylamine (DMAA) were mixed as the remainder (with respect to 100 wt % of the total amount of the composition/see Table 1), thereby preparing a composition.

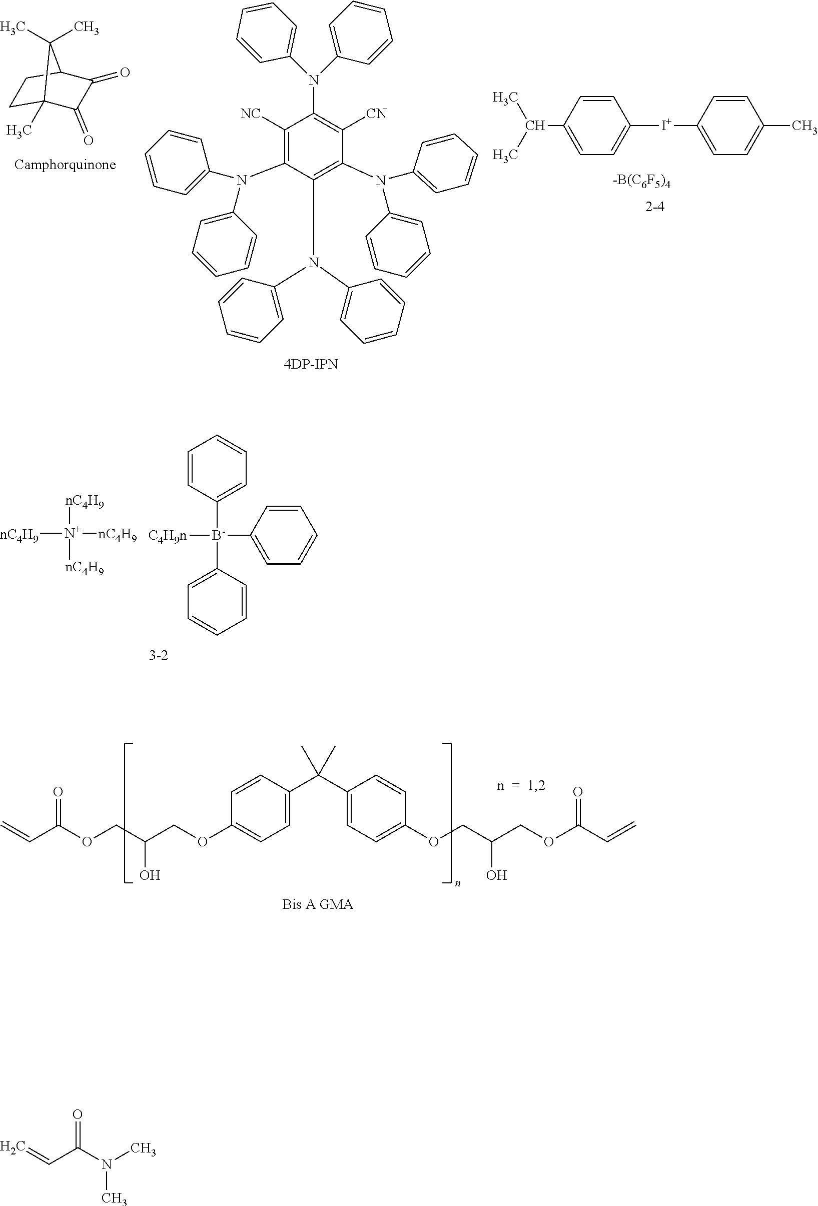

### DMAA

### Comparison of Conversion Rates of Polymers

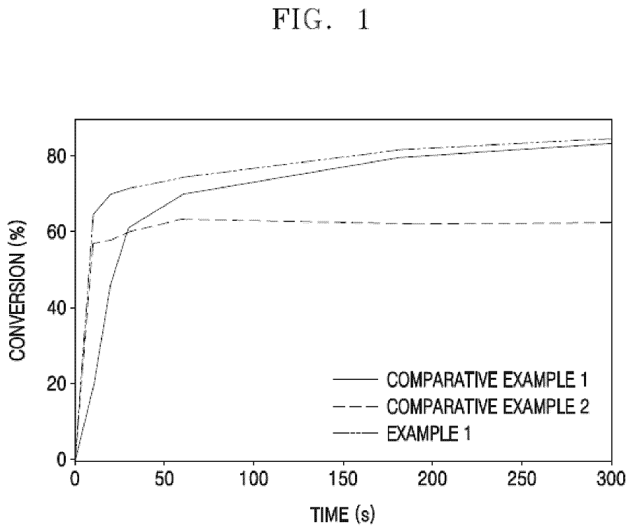

The compositions prepared in Comparative Example 1, Comparative Example 2, and Example 1 were applied onto a substrate to a thickness of 30 μm, and then each irradiated with light of 450 nm (100 MW/cm²) over time, and a degree of conversion of each formed polymer was measured and analyzed through Fourier transform infrared spectroscopy (FT-IR) and shown in FIG. 1.

Referring to FIG. 1, it can be seen that the polymer of Example 1 has a higher conversion rate than the polymers of Comparative Example 1 and Comparative Example 2.

A conversion rate when an irradiation time is 30 seconds is shown in Table 1 below.

TABLE 1

| | Initiator mixture | (ppm) | Conversion |
|---|---|---|---|
| Comparative | CQ | 4,000 | 61.35% |
| Example 1 | EDB | 24,000 | |
| Comparative | 4DP-IPN | 25 | 60.36% |
| Example 2 | Compound 2-4 | 3,000 | |
| | Compound 3-2 | 1800 | |
| Example 1 | CQ | 2,000 | 71.88% |
| | EDB | 12,000 | |
| | 4DP-IPN | 10 | |
| | Compound 2-4 | 3,000 | |
| | Compound 3-2 | 1,800 | |

Referring to Table 1, it can be seen that, in an initial stage of a curing reaction, the conversion rate of the polymer of Example 1 is significantly higher than that of the polymers of Comparative Example 1 and Comparative Example 2. Moreover, a content (e.g., amount) of the 4DP-IPN of Example 1 is lower than that of Comparative Example 2 by 50% or more, and a content (e.g., amount) of the CQ of Example 1 is lower than that of Comparative Example 1 by 50% or more.

### Deep Curing Evaluation

The compositions of Example 1, Comparative Example 1, and Comparative Example 2 were allowed to fill tubes having a certain standard and then cured over time at 480 nm (120 MW/cm²) to then compare curing depths.

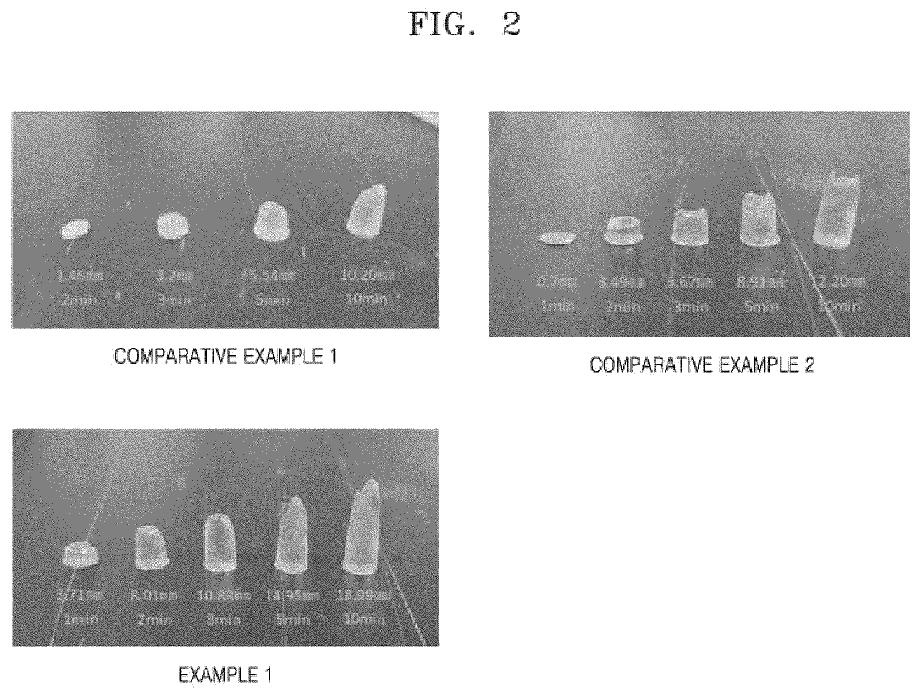

FIG. 2 shows images showing results of evaluating deep curing over time of the compositions of comparative examples and an example.

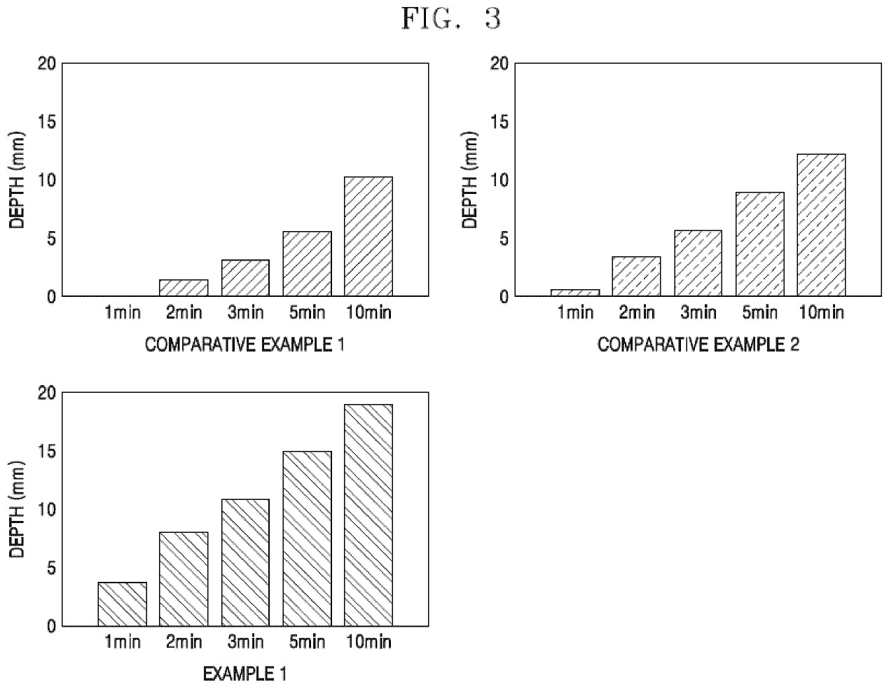

FIG. 3 shows graphs showing results of evaluating deep curing over time of the compositions of comparative examples and an example.

Referring to FIGS. 2 and 3, it can be seen that Example 1 has a deeper curing depth than Comparative Example 1 and Comparative Example 2.

From the above results, it is expected that a polymer formed from Example 1 will exhibit better physical properties than a polymer formed from Comparative Example 1 or Comparative Example 2.

A polymer formed using an initiator mixture according to an embodiment has a higher conversion rate and deeper curing depth than curing systems of a related art.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that various suitable changes in form and details may be made therein without departing from the spirit and scope of the present disclosure as defined by the following claims, and equivalents thereof.

What is claimed is:

1. An initiator mixture comprising:
a multi-aromatic ring-containing compound;
an ionic compound comprising iodine;
an ionic compound comprising boron;
a compound comprising a norbornene moiety which comprises an oxo group (O═); and
a tertiary amine compound comprising an aromatic moiety, an ester group, and an amine group.

2. The initiator mixture of claim 1, wherein the initiator mixture generates radicals in response to visible light.

3. The initiator mixture of claim 1, wherein the multi-aromatic ring-containing compound comprises a first compound represented by Formula 1 below: 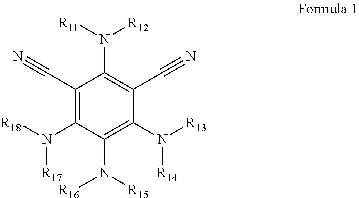
wherein R₁₁ to R₁₈ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more adjacent ones of R₁₁ to R₁₈ are optionally linked to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
R₁₀ₐ is: deuterium, —F, —Cl, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof; a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

4. The initiator mixture of claim 3, wherein the first compound is represented by Formula 1-1 or Formula 1-2 below: 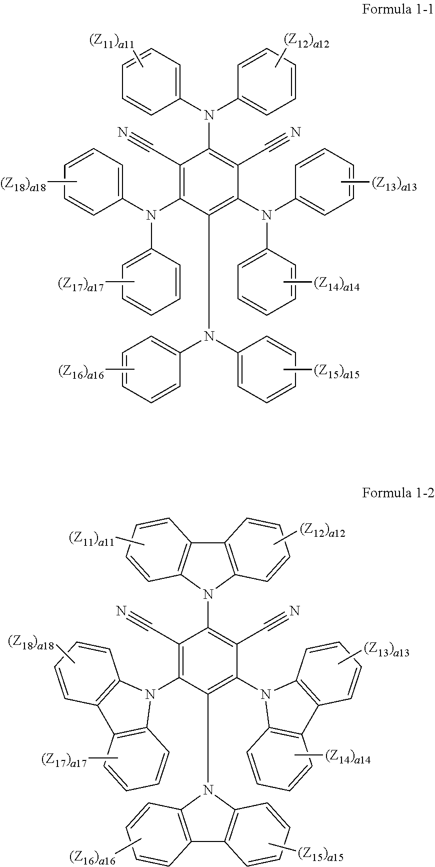
wherein, in Formula 1-1 and Formula 1-2,
Z₁₁ to Z₁₈ are each independently hydrogen or the same as defined for R₁₀ₐ in Formula 3, and
a11 to a18 are each independently an integer from 1 to 4.

5. The initiator mixture of claim 3, wherein the first compound comprises at least one selected from compounds below:

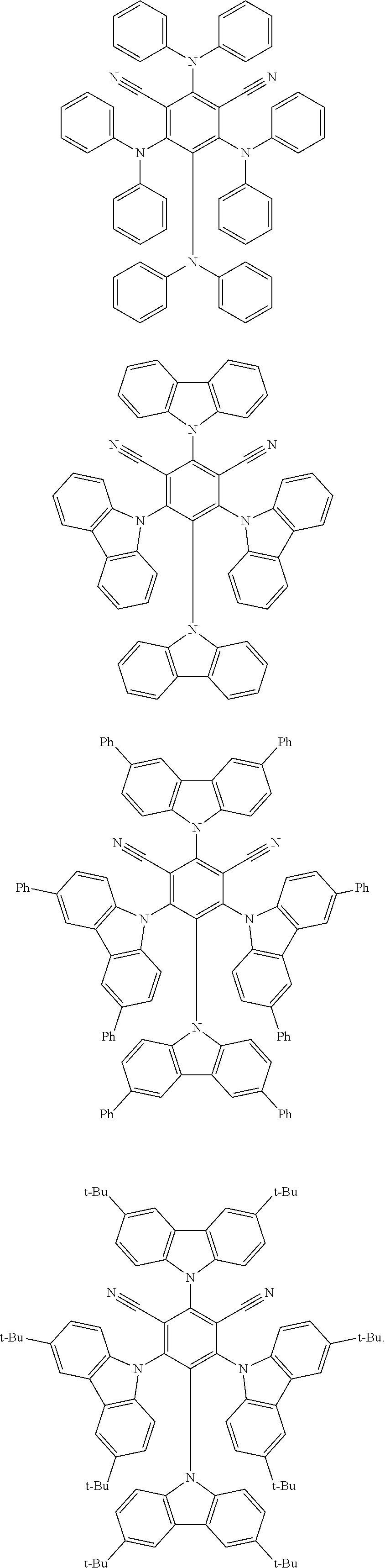

6. The initiator mixture of claim 1, wherein the ionic compound comprising iodine comprises a second compound represented by Formula 2: 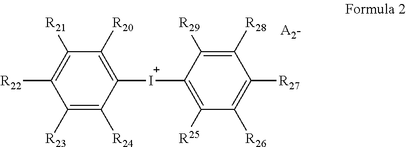
wherein, in Formula 2,
A₂_{^{−}} is a counter anion,
R₂₀ to R₂₉ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more adjacent ones of R₂₀ to R₂₉ are optionally linked to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
R₁₀ₐ is: deuterium, —F, —Cl, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof; a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

7. The initiator mixture of claim 6, wherein A₂_{^{−}} comprises a fluorine atom.

8. The initiator mixture of claim 1, wherein the ionic compound comprising iodine comprises at least one selected from compounds below:

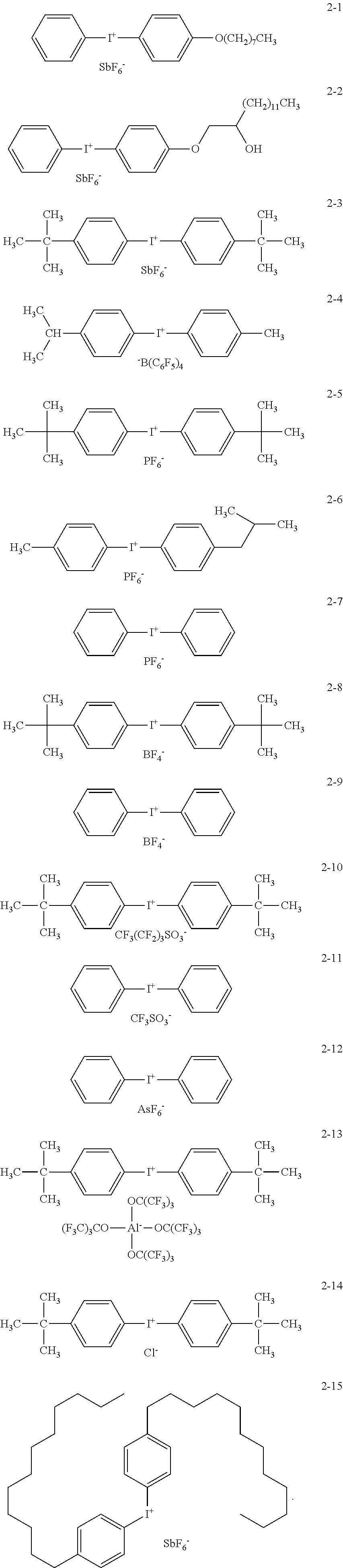

9. The initiator mixture of claim 1, wherein the ionic compound comprising boron comprises a third compound represented by Formula 3 below: 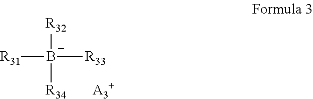
wherein, in Formula 3,
A₃_{⁺} is a counter cation,
R₃₁ to R₃₄ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
two or more adjacent ones of R₃₁ to R₃₄ are optionally linked to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
R₁₀ₐ is: deuterium, —F, —Cl, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof; a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

10. The initiator mixture of claim 9, wherein A₃_{⁺} is [P(R₃₁ₐ)(R_{31b})(R_{31c})(R_{31d})]⁺ or [N(R₃₂ₐ)(R_{32b})(R_{32c})(R_{32d})]⁺,
R₃₁ₐ, R_{31b}, R_{31c}, R_{31d}, R₃₂ₐ, R_{32b}, R_{32c}, and R_{32d} are each independently a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ, and
R₁₀ₐ is the same as defined for Formula 1.

11. The initiator mixture of claim 1, wherein the ionic compound comprising boron comprises at least one selected from compounds below:

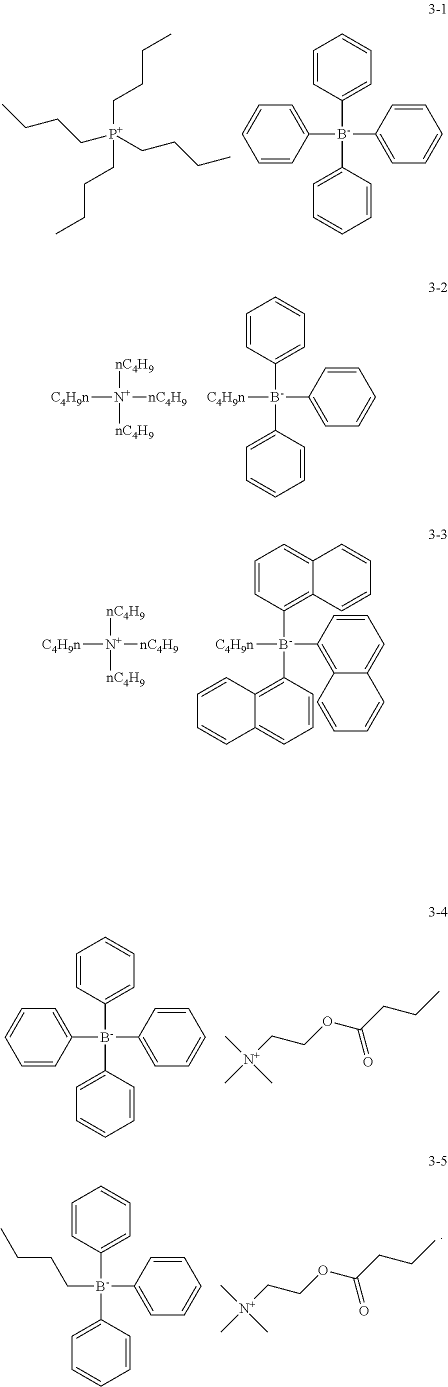

12. The initiator mixture of claim 1, wherein the norbornene moiety comprising an oxo group (O═) comprises 2-oxobicyclo[2.2.1]heptane.

13. The initiator mixture of claim 1, wherein the compound comprising the norbornene moiety which comprises an oxo group (O═) comprises camphorquinone, 7,7-dimethyl-2,3-dioxobicyclo[2.2.1]heptane-1-carboxylic acid, 7,7-dimethyl-2,3-dioxobicyclo[2.2.1]heptane-1-carboxy-2-bromoethyl ester, 7,7-dimethyl-2,3-dioxobicyclo[2.2.1]heptane-1-carboxy-2-methyl ester, 7,7-dimethyl-2,3-dioxobicyclo[2.2.1]heptane-1-carboxylic acid chloride, or any combination thereof.

14. The initiator mixture of claim 1, wherein the aromatic moiety of the tertiary amine compound comprises a phenyl group.

15. The initiator mixture of claim 1, wherein the aromatic moiety of the tertiary amine compound is a phenyl group, and
the ester group and the amine group are linked to the phenyl group and are at para positions of the phenyl group.

16. A composition comprising the initiator mixture of claim 1 and a monomer.

17. The composition of claim 16, wherein a content of the multi-aromatic ring-containing compound is greater than 0 parts per million (ppm) and less than or equal to 200 ppm with respect to a total amount of the composition.

18. The composition of claim 16, wherein a content of the ionic compound comprising iodine is in a range of about 500 parts per million (ppm) to about 6,000 ppm with respect to a total amount of the composition.

19. The composition of claim 16, wherein a content of the ionic compound comprising boron is in a range of about 600 part per million (ppm) to about 4,000 ppm with respect to a total amount of the composition.

20. A polymer formed by irradiating visible light onto the composition of claim 16.
